(19) United States
(12) Patent Application Publication
(10) Pub. No.: US 2024/0062890 A1
(43) Pub. Date: Feb. 22, 2024

(54) MOVEMENT HEALTH TRACKER USING A WEARABLE DEVICE

(71) Applicant: GOOGLE LLC, Mountain View, CA (US)

(72) Inventors: Dongeek Shin, San Jose, CA (US); Ricardo John Campbell, Redwood City, CA (US); Allison Yamanashi, Burlingame, CA (US)

(21) Appl. No.: 17/821,328

(22) Filed: Aug. 22, 2022

(51) Int. Cl. G16H 40/63; G16H 20/30

(52) U.S. Cl. CPC G16H 40/63; G16H 20/30

(57) ABSTRACT

A wearable device may be used to perform a health or medical assessment of a user. The wearable device may detect user movements of the user. The wearable device generates signal data representing the user movements. The signal data is input into a model to identify a feature set. The feature set is converted into a score that corresponds to a medical condition of the user.

### TECHNICAL FIELD

This description relates to a movement health tracker using a wearable device.

### BACKGROUND

When a person wants their movement health or motor health assessed, they schedule a visit to a doctor such as a neurologist. The neurologist may examine the person and perform one or more tests. For example, the Motor Examination section of the Unified Parkinson's Disease Rating Scale (UPDRS) specifies a few hand-based exercises for the person that can be observed and/or measured by the neurologist and that can be used to screen for and assess the motor signs of Parkinson's disease. The results of the hand-based exercises may be used by the neurologist to help diagnose the disease and/or track progress of the disease. While the neurologist may be able to diagnose and/or track progress of the disease, it may be difficult and challenging for the neurologist to precisely measure the features and/or details of the hand-based exercises and to accurately determine the results of the test just through visual observation of the person performing the test.

### SUMMARY

This document describes devices, systems, and techniques for providing a movement health tracker using a wearable device. For example, when a person wants their movement health or motor health assessed, the wearable device may be used to provide this assessment. In this manner, the person may be prompted, for example by the wearable device or by another device in communication with the wearable device, to perform an exercise, e.g., a hand-based exercise. The prompt may include instructions for how to perform the exercise. When the person performs the exercise, the wearable device detects user movements of the person wearing the wearable device. The wearable device includes a set of electronic sensors that are used to detect the user movements and to generate signal data representing the user movements. A model receives the signal data and identifies a feature set. The feature set is converted into a score that corresponds to a medical condition of the user.

In this manner, the wearable device allows for accurately measuring features and details of an exercise which is associated with a pre-defined physical test for determining a medical condition. The wearable device provides a technical solution for evaluating user movements and associate them with a medical condition. In this context, the proposed solution may for example allow to accurately measure timing and amplitude, and other features of user movements which have to be taken into account for assessing a medical condition based on the user movements performed. For example, more precise results related to the medical condition of the user can be provided by using generated signal data while the user preforms at least one exercise which the user is prompted to perform, and which is part of a pre-defined physical test for the medical condition.

Additionally, the use of the wearable device to provide the assessment of the medical condition of the user eliminates the inconvenience and necessity for making a physical visit to the doctor's office. The wearable device enables the user to perform, evaluate, and score the exercise without having to make an in-person visit to the doctor and without the direct administration and observation of the exercise by the doctor. The results, or the score corresponding to the medical condition of the user, may be communicated over a network to another device monitored by the doctor. In this manner, the user may perform the assessments in-home or at any location convenient for the user. Generally, the user movements may be part of at least one predetermined exercise the user is prompted to perform for generating the signal data and thus before generation of the signal data starts. The at least one exercise may have to be performed using an extremity on which the user wears the wearable device. For example, the exercise to be performed may be a hand-based exercise, i.e., an exercise to be performed using a hand of the user.

In one general aspect, a method includes detecting, by a wearable device worn by a user, user movements of the user; generating, by the wearable device, signal data representing the user movements; inputting the signal data into a model to identify a feature set; and converting the feature set into a score that corresponds to a medical condition of the user.

In another general aspect, a wearable device includes at least one memory, at least one processor coupled to the at least one memory, and a set of electronic sensors coupled to the at least one processor. The set of electronic sensors is configured to detect user movements of a user and to generate signal data representing the user movements. The at least one processor is configured to input the signal data into a model to identify a feature set and convert the feature set into a score that corresponds to a medical condition of the user.

In another general aspect, a non-transitory storage medium includes code that, when executed by processing circuitry, causes the processing circuitry to perform a method. The method includes detecting, by a wearable device worn by a user, user movements of the user; generating, by the wearable device, signal data representing the user movements; inputting the signal data into a model to identify a feature set; and converting the feature set into a score that corresponds to a medical condition of the user.

The details of one or more implementations are set forth in the accompanying drawings and the description below. Other features will be apparent from the description and drawings, and from the claims.

### BRIEF DESCRIPTION OF THE DRAWINGS

FIG. 1 is a diagram that illustrates an example wearable device used to generate signals for a medical assessment.

FIG. 2A is an example block diagram of the wearable device of FIG. 1.

FIG. 2B is an example block diagram of the computing device of FIG. 1.

FIG. 3 is a diagram that illustrates an example convolutional neural network (CNN) with stacked layers for different channels.

FIG. 4 is a flow chart that illustrates an example method using the wearable device and/or the computing device of FIG. 1.

### DETAILED DESCRIPTION

This disclosure describes devices and techniques to perform an assessment of a user for a medical condition using a wearable device. The wearable device may be worn on the arm or hand of the user and may prompt the user to initiate an assessment or a screening test by instructing the user to perform user movements. For example, the wearable device and/or a computing device in communication with the wearable device may instruct the user to perform particular user movements relevant to the assessment or screening for a medical condition. The wearable device processes the user movements and generates a score related to the assessment or screening for the medical condition. The score and/or a report related to the assessment or screening for the medical condition may then be output as a result. The results may be communicated and transmitted to the user's doctor, who may be in a location different from the user. In some implementations, the assessment or screening test may be a part of an ongoing assessment and/or a series of screening tests across a period of time such as, for example, a day, a week, a month, or other period of time. In this manner, the results of the ongoing assessment may be tracked over the period of time and the change in results may be recorded and/or reported.

The score indicative of the medical condition to be assessed may be based on a feature set identified on the basis of the signal data generated while the user performs a prompted exercise. For instances, the feature set may include at least one value determined to be characteristic for the signal data relating to the user movement. For example, the feature set may include at least one value relating to a time component and/or an amplitude component of the signal data. This may in particular relate to determine at least one value being indicative for a (local and/or global) peak in the generated signal data, in particular a time of occurrence of the peak in the signal data and/or an amplitude of the peak and using the at least one value for the feature set for conversion into the score indicative for the medical condition. For example, in a specified hand exercise, such as the finger tapping screening test, each event tap can be summarized with a two features output. The first is a time of a tap based on a local peak detection and the second is an amplitude of the tap. The feature set may be a feature bundle of a time series having the time of tap and the corresponding amplitude for each time of tap.

In this manner, the wearable device achieves the technical advantages and technical effects of self-monitoring and/or remote monitoring for a known or unknown medical condition. The technical solution includes using a set of electronic sensors on the wearable device to detect the user movements and generate signal data representative of the user movements. A model, such as a conventional neural network (CNN), is used to process the signal data and identify a feature set. Then, a feature analytics engine, which may include a decision tree and/or a regression network, may be used to convert the feature set to a score that corresponds to a medical condition of the user. The use of the wearable device and its components in this manner also provides a technical solution achieving more accurate tests and results compared to a visual observation and assessment of the test performed by the user in front of the doctor without the use of the wearable device.

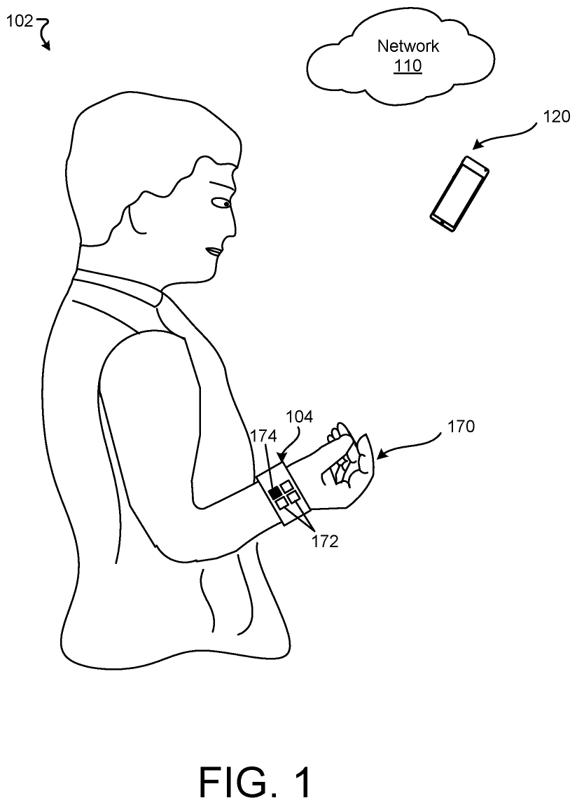

As shown in FIG. 1, a user 102 is wearing a wearable device 104 on their wrist. The wearable device 104 is configured to detect user movements (e.g., user gestures) of the user 102 and to generate signal data representing the user movements. As discussed in more detail below, the signal data is input into a model to identify a feature set. The feature set is converted into a score that corresponds to a medical condition of the user 102.

As shown in FIG. 1, the wearable device 104 takes the form of a wristband. The wearable device 104 can take other form factors such as, for example, a strap, a smartwatch, or an activity tracker, where all of the example forms of the wearable device 104 are configured to be worn on the wrist and to detect user movements of the user 102. In some implementations, the wearable device 104 may take other forms such as, for example, a ring that is configured to be worn on a finger and to detect user movements of the user 102.

The wearable device 104 includes a set of electronic sensors 172 and 174 (also referred to as electronic sensors 172 and 174). The electronic sensors 172 and 174, either individually or in combination, are configured to produce signal data representing user movements 170 of the user 102. That is, the electronic sensors 172 and/or 174 are configured to translate user movements 170 of the user 102, such a hand gesture formed by the user 102, into signal data. The signal data may thus include signal values and/or gradients which are characteristic for the user movement performed, e.g., with respect to timing(s) and amplitude(s). As mentioned above and as discussed in more detail below, this signal data is used in determining a medical condition or medical status of the user 102.

In some implementations, the sensors 172 include inertial measurement units (IMUs). An IMU is a device that includes a combination of accelerometers, gyroscopes, and in some cases, magnetometers, in order to measures and reports an acceleration and, in some cases, an orientation.

In some implementations, the sensors 174 include a photoplethysmography (PPG) sensor. The PPG sensor is an optical sensor configured to detect and measure hand micromovements via exposing small arterial volume changes to optical radiation. The PPG sensor includes one or more illuminators (e.g., LEDs) and one or more detectors (e.g., photodiodes). The LEDs can be configured to transmit focused light towards a user's wrist. The transmitted light may include wavelengths in the visible portion of the spectrum (e.g., 530 nanometer (green)) for increased resolution (i.e., visible wavelength) and/or wavelengths in the infrared portion of the spectrum (e.g., 730 nanometer (nm)) for increased skin penetration (i.e., infrared wavelength). For example, the wavelength may be in a near infrared (NIR) portion of the section.

The transmitted light can penetrate the skin of the user to illuminate blood vessels of the user 102. Blood in the blood vessels can reflect (i.e., back-reflect) light towards the photodiodes. The photodiodes are directed to the wrist of the user 102 to measure an intensity of the back-reflected light. The intensity of the back-reflected light is modulated as the volume of the blood in the blood vessels change. Accordingly, signals from the photodiodes may be processed (e.g., filtered) and analyzed (e.g., Fourier transformed) to determine user movements as well as other information such as, for example, a heart rate. The processing may include low-pass filtering of the back-reflected light to obtain frequencies corresponding to the user movements, which may be in a relatively low frequency band. Including PPG sensors 174 may make the signal data more robust and assist in avoiding false positives.

In some implementations, the wearable device 104 includes in sensors 172 and/or 174 and a compass. The compass is configured to measure an absolute orientation and provide absolute orientation data in another signal. The absolute orientation may provide additional context as to the orientation of the user's hand as it makes user movements 170 (such as a hand gesture).

During operation, the user 102 may be prompted to perform the user movement 170 that will be used to assess the user's medical condition. For example, the wearable device 104 may provide a visual and/or audio prompt that instructs the user 102 to perform the user movement 170. In some implementations, the computing device 120 may provide a visual and/or audio prompt that instructs the user 102 to perform the user movement 170. Upon performing the user movement 170, the electronic sensors 172 and/or 174 detect the user movement 170 and generate signal data representing the user movement 170.

In some implementations, the user movement 170 may be a prescribed assessment related to a particular medical condition. For example, the user 102 may be prompted to perform a hand exercise as prescribed in the motor examination section of the Unified Parkinson's Disease Rating Scale (UPDRS). One example hand exercise or test may be finger tapping. The user 102 is prompted to use the hand wearing the wearable device 104 and tap the index finger on the thumb 10 times as quickly and as big as possible. The user 102 performs the gesture with their hand. Upon performing the user movement 170, the user's wrist muscles will move in specific ways, based on the movement of the user's hand in making the user movement 170. The wearable device 104, upon sensing wrist muscle movement, performs measurements using the IMU sensors 172 and PPG sensors 174. Each of the IMU and PPG sensors 172 and 174 then generate signal data in the form of a time series. Further details concerning the signals are discussed below. In some implementations, the assessment or screening test for Parkinson's disease may be a part of an ongoing assessment and/or a series of screening tests across a period of time such as, for example, a day, a week, a month, or other period of time. In this manner, the results of the ongoing assessment may be tracked over the period of time and the change in results may be recorded and/or reported, as related to tracking the change in the medical condition of the user for Parkinson's disease.

In some implementations, other assessments and/or screening tests could be performed using the wearable device 104 to assess a user's health movement related to other medical conditions such as, for example, Multiple Sclerosis (MS), Essential Tremor (ET), Multiple System Atrophy, and other diseases.

The electronic sensors 172 and/or 174 detect the user movement 170 and generates signal data representing the user movement 170. The signal data may be stored in memory on the wearable device 104 and/or the computing device 120. The signal data may be input into a model to generate a feature set and the feature set may be converted into a score that corresponds to a medical condition of the user 102. For instances, the feature set may include at least one value determined to be characteristic for the signal data relating to the user movement. For example, the feature set may include at least one value relating to a time component and/or an amplitude component of the signal data. This may in particular relate to determine at least one value being indicative for a (local and/or global) peak in the generated signal data, in particular a time of occurrence of the peak in the signal data and/or an amplitude of the peak and using the at least one value for the feature set for conversion into a score indicative for the medical condition. For example, in a specified hand exercise, such as the finger tapping screening test, each event tap can be summarized with a two features output. The first is a time of a tap based on a local peak detection and the second is an amplitude of the tap. The feature set may be a feature bundle of a time series having the time of tap and the corresponding amplitude for each time of tap. For instance, the score may correspond to a scale related to Parkinson's disease, where “0” is normal, “1” is slight, “2” is mild, “3” is moderate, and “4” is severe. Each of the particular ratings has a definition related to the finger tapping exercise. A report may be generated for the user 102 on the wearable device 104 and/or on the computing device 120.

After performing the test using the one hand wearing the wearable device 104, the user 102 may be prompted to move the wearable device 104 to the other wrist and to perform the test again so that each hand may be evaluated separately.

In some implementations, the signal data may be transmitted over a network 110 to a computing device 120. As shown in FIG. 1, the computing device 120 is a mobile phone. In some implementations, however, the computing device 120 is a desktop, laptop, tablet, server, or the like, as long as the computing device 120 is configured to receive data from signals.

In some implementations, the network 110 is a wireless network configured to transmit signal data generated by the wearable device 104 to the computing device 120. In some implementations, the wireless network includes a WiFi network. In some implementations, the network 110 includes a wireless radio. In some implementations, the wireless radio is one of LTE, LTE-A, 5G (New Radio, or NR), cmWave, and/or mmWave band networks, or any other wireless network.

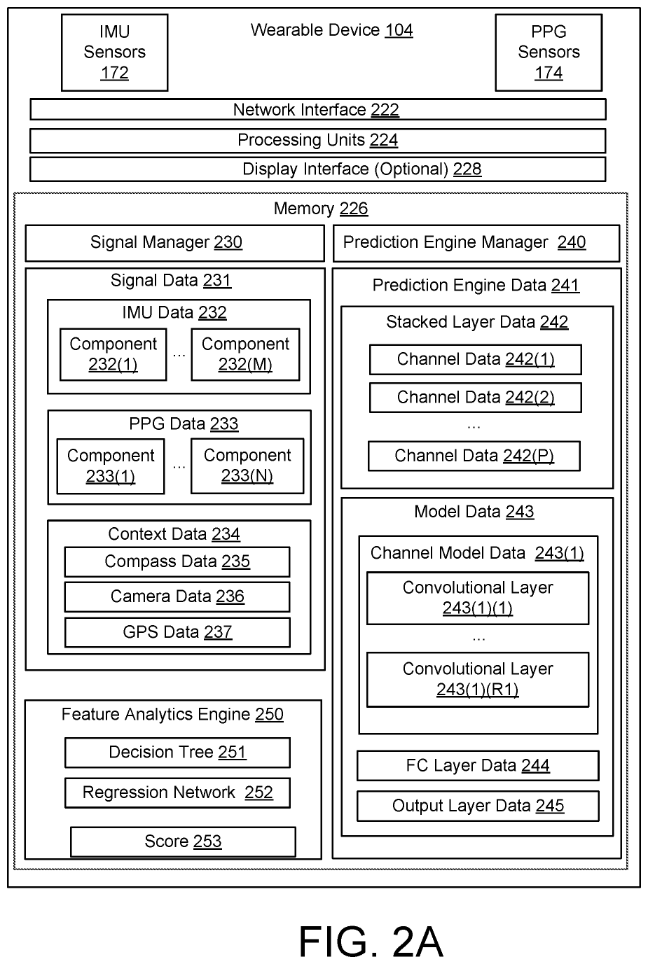

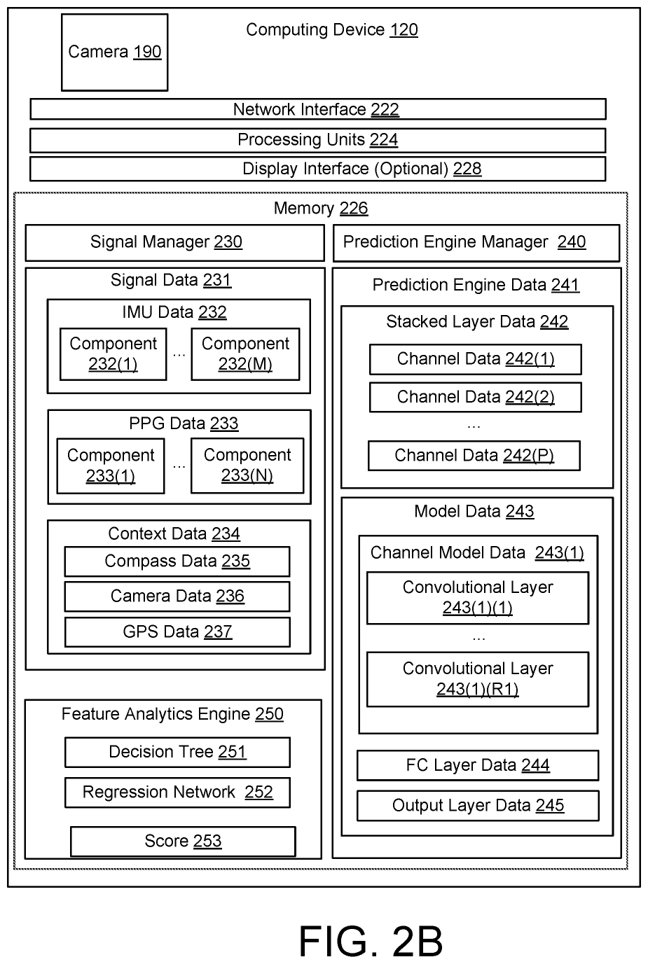

With continued reference to FIG. 1, once the signal data is generated, the signal data is input into a model to identify a feature set. Then, the feature set is processed by a feature analytics engine and the feature set is converted into the score that corresponds to the medical condition of the user. In some implementations, the model and the feature analytics engine are implemented on the wearable device 104. In some implementations, the model and the feature analytics engine are implemented on the computing device 120. In some implementations, the model and the feature analytics engine may be implemented on both the wearable device 104 and the computing device 120. In some implementations, the model is implemented on one of the devices and the feature analytics engine is implemented on the other device. The computing device 120 is configured to receive signal data generated by the wearable device 104 and apply a model (e.g., a convolutional neural network (CNN)) to the received signals to generate a feature set. Further details regarding the wearable device 104 are described in FIG. 2A and further details regarding the computing device 120 are described in FIG. 2B.

FIG. 2A is an example block diagram of the wearable device 104. The wearable device 104 includes the IMU sensors 172 and the PPG sensors 174. The wearable device 104 includes a network interface 222, processing units 224, a memory 226, and a display interface (optional) 228. As discussed above, the IMU sensors 172 and/or the PPG sensors 174 detect user movement of the user and generate signal data 231 representative of the user movement of the user.

The network interface 222 includes, for example, Ethernet adaptors, Token Ring adaptors, and the like, for converting electronic and/or optical signals received from the network to electronic form for use by the wearable device 104. The set of processing units 224 include one or more processing chips and/or assemblies. The memory 226 may include both volatile memory (e.g., RAM) and non-volatile memory, such as one or more ROMs. The set of processing units 224 and the memory 226 together form controlling circuitry, which is configured and arranged to carry out various methods and functions as described herein. The memory 226 may be an example of a non-transitory computer-readable medium or a non-transitory computer-readable storage medium.

In some implementations, one or more of the components of the wearable device 104 can be, or can include processors (e.g., processing units 224) configured to process instructions stored in the memory 226. Examples of such instructions as depicted in FIG. 2A include a signal manager 230, a prediction engine manager 240, and a feature analytics engine 250. Further, as illustrated in FIG. 2A, the memory 226 is configured to store various data, which is described with respect to the respective managers that use such data.

The signal manager 230 is configured to obtain signal data 231. For example, in response to a gesture or hand exercise performed by the user, the wearable device 104, via the IMU sensors 172 and/or the PPG sensors 174, generates signals representative of the gesture or hand exercises. The signal manager 230 extracts data carried by the signals and arranges the signal data 231 as shown in FIG. 2A.

The signal data 231 represents information about gestures formed by the user 102 from a feature set may be deduced by a model. The arrangement of the signal data 231a as shown in FIG. 2A is designed for a custom model. As shown in FIG. 2A, the signal data includes IMU data 232, PPG data 233, and context data 234.

The IMU data 232 represents signal data as generated by an IMU sensor 172. As shown in FIG. 2A, IMU data 232 includes components 232(1-M). In some implementations, the components 232(1-M) represent spatial components of the IMU signal (i.e., x, y, z); in this case M=3. In some implementations, the components 232(1-M) represent acceleration data in (x, y, and z) directions.

Returning to FIG. 2A, the PPG data 233 represents signal data as generated by a PPG sensor 174. As shown in FIG. 2A, PPG data 233 includes components 233(1-M). In some implementations, the components 233(1-M) represent spatial components of the PPG signal (i.e., x, y, z); in this case M=3. In some implementations, the components 233(1-M) represent acceleration data in (x, y, and z) directions.

In some implementations, the context data 234 represents additional signal information. The context data 234, in concert with the IMU data 232 and PPG data 233, may provide more robustness of the feature set. As shown in FIG. 2A, the context data 234 includes compass data 235, camera data 236, and GPS data 237. In some implementations, the context data 234 includes one or two of the data 235, camera data 236, and GPS data 237.

The compass data 235 represents an absolute orientation of the hand of the user 102 which performs the user movement 170. In some implementations, the compass that generates the compass data 235 is included in the wearable device 104.

The camera data 236 represents an image of the user movement 170 formed by the user 102. The camera data 236 may be formed by a camera on the computing device 120 that is transmitted to the wearable device 104. The camera data 236 may be useful in, for example, determining orientations of the user movements and/or verification of the user movements.

The GPS data 237 represents a location of the user. In some implementations, the GPS data is generated by a GOS device built into the wearable device 104.

The prediction engine manager 240 is configured to arrange the signal data 231 into channels within prediction engine data 241 and generate the feature set. The prediction engine manager 240 is also configured to generate separate models for each of the channels; in this respect, the prediction engine manager 240 is configured to train each of the models based on user movement data from a population of users. The prediction engine manager 240 is configured to combine the output from each of these models to produce combined data forming the feature set.

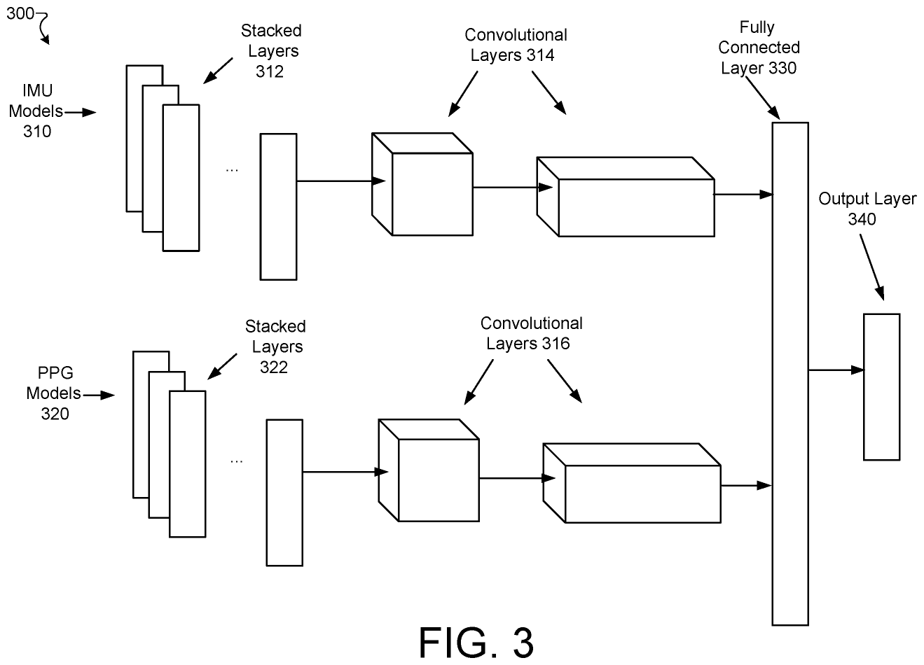

The prediction engine data 241 represents the inputs, model parameter values, and outputs used and generated by the prediction engine manager 240a. The models trained and evaluated by the prediction engine manager 240 are convolutional neural networks (CNNs). Before describing the elements of the models, the overall model is described with regard to FIG. 3.

FIG. 3 is a diagram that illustrates an example CNN 300 with stacked layers as model input for different channels. As shown in FIG. 3, the CNN model 300 places each signal component from each signal source in stacked layers. For example, the IMU models 310 input each spatial component of the IMU signal into stacked layers 312 and the PPG models 320 input each component of the PPG signal into stacked layers 322. That is, data from the channel containing the x-acceleration signal is put into an input layer of a dedicated CNN model for the IMU x-acceleration. The data from the channels containing the IMU y- and z-acceleration signals, respectively, is similarly put into input layers of dedicated CNN models for the IMU y- and z-acceleration. PPG signal components are similarly put into input layers of their respective dedicated CNN models.

As shown in FIG. 3, the input data in the IMU stacked layers 312 are fed into intermediate, hidden convolutional layers 314. Similarly, the input data in the PPG stacked layers 322 are fed into intermediate, hidden convolutional layers 316. Again, each signal component from each device is processed in its own, respective model. Using separate models in this fashion enhances the accuracy of the predictions.

Also as shown in FIG. 3, the values of the final convolutional layers 314 and 316 are then input into a fully connected layer 330. In the fully connected layer 330, the values of each signal component from each source are combined to produce a single set of values to be input into an output layer 340. From these single values, the overall model outputs a feature set. The feature set includes a time component and an amplitude component. For example, in a specified hand exercise such as the finger tapping screening test each event tap can be summarized with two features output by the output layer 340. The first is a time of tap based on the local peak detection and the second is an amplitude of the tap. The feature set may be a feature bundle of a time series having the time of tap and the corresponding amplitude for each time of tap.

Returning to FIG. 2A, the prediction engine data 241a includes stacked layer data 242 and model data 243. The stacked layer data 242 represents the signal components from each channel corresponding to a respective signal source (e.g., IMU, PPG, context). Each channel of the stacked layer data 242 is input into its own respective model represented by model data 243. As shown in FIG. 2A, the stacked layer data 242 includes channel data 242(1-P), where P is the number of signal components and sources.

Each channel data 242(1-P), e.g., channel data 242(1), represents an amplitude and/or phase of a signal component from a sensor. That is, channel data 242(1) may represent an IMU x-acceleration, channel data 242(2) represents an IMU y-acceleration, and so on. Some channel data, e.g., 242(4) may represent a PPG signal component. Nevertheless, in some implementations, each channel data 242(1-P) includes streaming values that form a time series.

The model data 243 represents data defining the channel models 243(1-P) corresponding to each of the channel data 242(1-P). Each model, e.g., 243(1) includes parameter values corresponding to each convolutional layer 243(1)(1-R1) in each model, where R1 is the number of convolutional layers in the model corresponding to channel data 242(1). In some implementations, the number of parameters is less than 10,000. Moreover, each model, e.g., 243(1) may or may not include pooling layers, skip layers, and nonlinear activation functions. In some implementations, the models are trained in a supervisor framework using a loss function based on a difference between predicted results and actual results.

As shown in FIG. 2A, the prediction engine data 241 also includes fully connected layer data 244 and output layer data 245. The fully connected layer data 244 is configured to take in the outputs of each channel model represented by channel model data 243(1-P), i.e., the values in convolution layers 243(1)(R1)-243(P)(RP), and combine them to produce values for the output layer data 245. In some implementations, the combining of the values in convolution layers 243(1)(R1)-243(P)(RP) is a concatenation of those values, i.e., the output of each convolutional layer are stacked end-to-end to form the fully connected layer. In some implementations, the results are averaged. In some implementations, the averaging is weighted based on a criterion.

The feature analytics engine 250 is configured to convert the feature set output from the output layer data 245 into a score 253. In some implementations, the feature analytics engine 250 may include a hardcoded decision tree 251 to map the feature set to one of the score buckets. For example, as discussed above with respect to the finger tapping score, the feature analytics engine 250 may include a hardcoded decision tree 251 to map the feature set to one of the score buckets −0, 1, 2, 3, or 4. For example, if the user is only able to complete a couple of finger taps in a given time period (e.g., 10 seconds), then the feature analytics engine may include a decision tree 251 that maps this number of taps to a score of “2”, according to the UPDRS guidelines.

In some implementations, the feature analytics engine 250 may include a regression network 252, such as a fully connected regression network, that is configured to process the feature set to the score 253. One advantage of using the regression network 252 is that it has the potential for higher accuracy compared to the decision tree 251 and also has a continuous value output when the regression network 252 is trained with integer outputs but regressed on them.

Referring to FIG. 2B, an example block diagram of the computing device 120 is illustrated. The computing device 120 may include the same or similar components as the wearable device 104. For example, the computing device 120 may include the network interface 222, the processing units 224, the memory 226, the display interface 228, the signal manager 230, the prediction manage 240, and the feature analytics engine 250. These components includes the same features and functionality as described above with respect to the wearable device 104. The computing device 120 may include a camera 190.

The components (e.g., modules, processing units 224) of the wearable device 104 and the computing device 120 can be configured to operate based on one or more platforms (e.g., one or more similar or different platforms) that can include one or more types of hardware, software, firmware, operating systems, runtime libraries, and/or so forth. In some implementations, the components of the computing device 120 can be configured to operate within a cluster of devices (e.g., a server farm). In such an implementation, the functionality and processing of the components of the computing device 120 can be distributed to several devices of the cluster of devices.

The components of the wearable device 104 and the computing device 120 can be, or can include, any type of hardware and/or software configured to process attributes. In some implementations, one or more portions of the components shown in the components of the wearable device 104 and the computing device 120 can be, or can include, a hardware-based module (e.g., a digital signal processor (DSP), a field programmable gate array (FPGA), a memory), a firmware module, and/or a software-based module (e.g., a module of computer code, a set of computer-readable instructions that can be executed at a computer). For example, in some implementations, one or more portions of the components of the wearable device 104 and the computing device 120 can be, or can include, a software module configured for execution by at least one processor (not shown). In some implementations, the functionality of the components can be included in different modules and/or different components than those shown in FIGS. 2A and 2B, including combining functionality illustrated as two components into a single component.

Although not shown, in some implementations, the components of the computing device 120 (or portions thereof) can be configured to operate within, for example, a data center (e.g., a cloud computing environment), a computer system, one or more server/host devices, and/or so forth. In some implementations, the components of the computing device 120 (or portions thereof) can be configured to operate within a network. Thus, the components of the computing device 120 (or portions thereof) can be configured to function within various types of network environments that can include one or more devices and/or one or more server devices. For example, the network can be, or can include, a local area network (LAN), a wide area network (WAN), and/or so forth. The network can be, or can include, a wireless network and/or wireless network implemented using, for example, gateway devices, bridges, switches, and/or so forth. The network can include one or more segments and/or can have portions based on various protocols such as Internet Protocol (IP) and/or a proprietary protocol. The network can include at least a portion of the Internet.

As illustrated in FIGS. 2A and 2B, the memory 226 is configured to store various data, including signal data 231 including IMU data 232, PPG data 233 and context data 234, prediction engine data 241 including stacked layer data 242, model data 243, FC layer data 244, and output layer data 245.

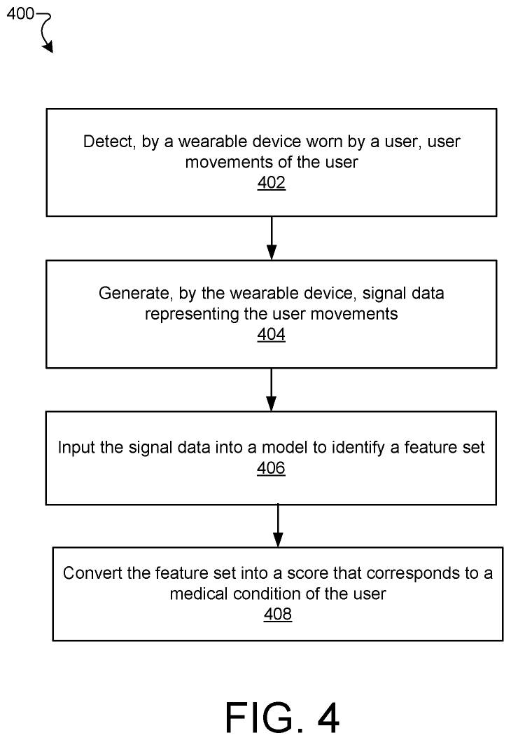

FIG. 4 is a flow chart depicting an example method 400. The method 400 may be performed by software constructs described in connection with FIGS. 2A and 2B, which reside in memory 226 of the wearable device 104 and/or the computing device 120 and are run by the set of processing units 224.

Process 400 includes detecting, by a wearable device worn by a user, user movements of the user (402). For example, the wearable device 104 may detect user movements of the user. More specifically, the set of electronics including the IMU sensor 172 and/or the PPG sensor 174, may detect user movements of the user.

In some implementations, the user may be prompted, in particular visually and/or audibly instructed to perform the user movements that include a gesture designed to assess a specific medical condition. For example, the user may be prompted by the wearable device 104 and/or the computing device 120 to perform a hand exercise such as the finger tapping exercise described above to assess the user for Parkinson's disease.

Process 400 includes generating, by the wearable device, signal data representing the user movements (404). For example, the wearable device 104 may generate the signal data representing the user movements. More specifically, the set of electronics including the IMU sensor and/or the PPG sensor 174, may generate the signal data representing the user movements, i.e., signal data generated while the users performs the prompted movement and thus being characteristic for the movements as performed by the individual user.

Process 400 includes inputting the signal data into a model to identify a feature set (406). For example, the wearable device 104 may input the signal data into a model to identify the feature set. In some implementations, the model is a CNN that includes stacked layers, where each of the layers corresponds to a respective electronic sensor of the set of electronic sensors. In some implementations, the model is implemented on the wearable device 104. In some implementations, the model is implemented on the computing device 120 and the wearable device 104 transmits the signal data to the computing device 120.

In some implementations, the feature set includes a time series having two features. The two features include a time and an amplitude corresponding to the time. In the example of the finger tapping assessment, the time is the time of the tap of the index finger to the thumb and the amplitude is a value of the force of the tap between the index finger and the thumb. The feature set may be measured over a period of time such as, for example, less than 30 seconds, e.g., 10, 15, or 20 seconds. Other time periods may be used or as designated by a particular screening test or assessment.

Process 400 includes converting the feature set into a score that corresponds to a medical condition of the user (408). For example, the wearable device 104 and/or the computing device 120 may convert the feature set into a score that corresponds to a medical condition of the user. More specifically, a feature analytics engine 250 may be configured to convert the feature set into a score that corresponds to a medical condition of the user. In some implementations, the feature analytics engine 250 may include a decision tree 251 that converts the feature set into the score. In some implementations, the feature analytics engine 250 may include a regression network 252 that converts the feature set into the score.

In the example of the finger tapping assessment, the time series feature set of time and corresponding amplitudes is converted to a score that corresponds to the scoring for Parkinson's disease screening as outlined by the UPDRS.

In some implementations, the score may be displayed and a report may be generated for display to the user on the wearable device 104 and/or the computing device 120. In some implementations, the score and/or the report may be transmitted to the user's doctor such that the doctor is made aware of the results of the assessment. In this manner, the user may not need to go in-person to the doctor's office to perform the assessment in front of the doctor. Instead, the user may perform the assessment from practically any location and environment and have the results of the assessment transmitted to their doctor.

In the following, some examples are described.
Example 1: A method including detecting, by a wearable device worn by a user, user movements of the user; generating, by the wearable device, signal data representing the user movements; inputting the signal data into a model to identify a feature set; and converting the feature set into a score that corresponds to a medical condition of the user.
Example 2: The method as in example 1, where the wearable device includes a wristband.
Example 3: The method as in example 1 or 2, where the model includes a convolutional neural network (CNN) configured to receive the signal data as input and to identify the feature set as output.
Example 4: The method of any of the preceding examples, where the wearable device worn by the user includes a set of electronic sensors, each of the set of electronic sensors being configured to produce the signal data in response to detecting the user movements.
Example 5: The method as in example 4, where the set of electronic sensors comprises an inertial measurement unit (IMU) sensor.
Example 6: The method as in example 4 or 5, where the set of electronic sensors includes a photoplethysmography (PPG) sensor.
Example 7: The method as in any of examples 3 through 6, where the CNN includes a plurality of stacked layers, each of the plurality of stacked layers corresponding to a respective electronic sensor of the set of electronic sensors.
Example 8: The method as in any of the preceding examples, where converting the feature set includes converting the feature set, by a feature analytics engine, into the score that corresponds to the medical condition of the user.
Example 9: The method as in example 8, where the feature analytics engine includes a fully connected regression network configured to convert the feature set into the score.
Example 10: The method as in example 8 or 9, where the feature analytics engine includes a decision tree configured to map the feature set into the score.
Example 11: The method as in any of the preceding, where the user movements include finger tapping of a thumb against an index finger on a same hand of the user on which the wearable device is worn and the score corresponds to a rating on a unified Parkinson's disease rating scale.
Example 12: A wearable device includes at least one memory, at least one processor coupled to the at least one memory, and a set of electronic sensors coupled to the at least one processor. The set of electronic sensors is configured to detect user movements of a user and to generate signal data representing the user movements. The at least one processor is configured to input the signal data into a model to identify a feature set and to convert the feature set into a score that corresponds to a medical condition of the user.
Example 13: The wearable device of example 12, where the wearable device comprises a wristband.
Example 14: The wearable device of example 12 or 13, where the model includes a convolutional neural network (CNN) configured to receive the signal data as input and to identify the feature set as output.
Example 15: The wearable device of example 14, where the set of electronic sensors includes an inertial measurement unit (IMU) sensor.
Example 16: The wearable device of example 14 or 15, where the set of electronic sensors includes a photoplethysmography (PPG) sensor.
Example 17: The wearable device of any of examples 12 through 16, where converting the feature set includes converting the feature set, by a feature analytics engine, into the score that corresponds to the medical condition of the user.
Example 18: The wearable device of example 17, where the feature analytics engine includes a fully connected regression network configured to convert the feature set into the score.
Example 19: The wearable device of example 17 or 18, where the feature analytics engine includes a decision tree configured to map the feature set into the score.
Example 20: The wearable device of any of examples 12 through 19, where the user movements include finger tapping of a thumb against an index finger on a same hand of the user on which the wearable device is worn and the score corresponds to a rating on a unified Parkinson's disease rating scale.
Example 21: A non-transitory storage medium includes code that, when executed by processing circuitry, causes the processing circuitry to perform a method. The method includes detecting, by a wearable device worn by a user, user movements of the user; generating, by the wearable device, signal data representing the user movements; inputting the signal data into a model to identify a feature set; and converting the feature set into a score that corresponds to a medical condition of the user.
Example 22: The non-transitory storage medium of example 21, where the wearable device comprises a wristband.
Example 23: The non-transitory storage medium of example 21 or 22, where the model includes a convolutional neural network (CNN) configured to receive the signal data as input and to identify the feature set as output.
Example 24: The non-transitory storage medium of any of examples 21 through 23, where the wearable device worn by the user includes a set of electronic sensors, each of the set of electronic sensors being configured to produce the signal data in response to detecting the user movements.
Example 25: The non-transitory storage medium of example 24, where the set of electronic sensors includes an inertial measurement unit (IMU) sensor.
Example 26: The non-transitory storage medium of example 24 or 25, where the set of electronic sensors includes a photoplethysmography (PPG) sensor.
Example 27: The non-transitory storage medium of any of examples 21 through 26, where converting the feature set includes converting the feature set, by a feature analytics engine, into the score that corresponds to the medical condition of the user.
Example 28: The non-transitory storage medium of example 27, where the feature analytics engine includes a fully connected regression network configured to convert the feature set into the score.
Example 29: The non-transitory storage medium of example 27 or 28, where the feature analytics engine includes a decision tree configured to map the feature set into the score.
Example 30: The non-transitory storage medium of any of examples 21 through 29, where the user movements include finger tapping of a thumb against an index finger on a same hand of the user on which the wearable device is worn and the score corresponds to a rating on a unified Parkinson's disease rating scale.
Example 31: The non-transitory storage medium of example 21, where the wearable device includes a wristband, the model includes a convolutional neural network (CNN) configured to receive the signal data as input and to identify the feature set as output, the wearable device includes a set of electronic sensors, each of the set of electronic sensors being configured to produce the signal data in response to detecting the user movements, and the set of electronic sensors includes an inertial measurement unit (IMU) sensor and a photoplethysmography (PPG) sensor.
Example 32: A method including means for detecting, by a wearable device worn by a user, user movements of the user; means for generating, by the wearable device, signal data representing the user movements; means for inputting the signal data into a model to identify a feature set; and means for converting the feature set into a score that corresponds to a medical condition of the user.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor and can be implemented in a high-level procedural and/or object-oriented programming language, and/or in assembly/machine language.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

A number of implementations have been described. Nevertheless, it will be understood that various modifications may be made without departing from the spirit and scope of the specification.

It will also be understood that when an element is referred to as being on, connected to, electrically connected to, coupled to, or electrically coupled to another element, it may be directly on, connected or coupled to the other element, or one or more intervening elements may be present. In contrast, when an element is referred to as being directly on, directly connected to or directly coupled to another element, there are no intervening elements present. Although the terms directly on, directly connected to, or directly coupled to may not be used throughout the detailed description, elements that are shown as being directly on, directly connected or directly coupled can be referred to as such. The claims of the application may be amended to recite example relationships described in the specification or shown in the figures.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the implementations. It should be understood that they have been presented by way of example only, not limitation, and various changes in form and details may be made. Any portion of the apparatus and/or methods described herein may be combined in any combination, except mutually exclusive combinations. The implementations described herein can include various combinations and/or sub-combinations of the functions, components and/or features of the different implementations described.

In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other implementations are within the scope of the following claims.

What is claimed is:

1. A method comprising:
detecting, by a wearable device worn by a user, user movements of the user;
generating, by the wearable device, signal data representing the user movements;
inputting the signal data into a model to identify a feature set; and
converting the feature set into a score that corresponds to a medical condition of the user.

2. The method as in claim 1, wherein the user movements are part of at least one predetermined exercise the user is prompted to perform before generating the signal data.

3. The method as in claim 2, wherein the at least one predetermined exercise the user is prompted to perform comprises at least one exercise to performed with an extremity of the user on which the wearable device is worn.

4. The method as in claim 3, the method further comprising generating the signal data while the user performs the at least one exercise with the extremity of the user on which the wearable device is worn.

5. The method as in claim 1, wherein:
the user movements include finger tapping of a thumb against an index finger on a same hand of the user on which the wearable device is worn.

6. The method as in claim 5, wherein the score corresponds to a rating on a unified Parkinson's disease rating scale.

7. The method as in claim 1, wherein the wearable device comprises a wristband.

8. The method as claim 1, wherein the model comprises a convolutional neural network (CNN) configured to receive the signal data as input and to identify the feature set as output.

9. The method as in claim 8, wherein the wearable device worn by the user includes a set of electronic sensors, each of the set of electronic sensors being configured to produce the signal data in response to detecting the user movements.

10. The method as in claim 9, wherein the set of electronic sensors comprises an inertial measurement unit (IMU) sensor.

11. The method as in claim 10, wherein the set of electronic sensors includes a photoplethysmography (PPG) sensor.

12. The method as in claim 11, wherein the CNN includes a plurality of stacked layers, each of the plurality of stacked layers corresponding to a respective electronic sensor of the set of electronic sensors.

13. The method as in claim 1, wherein converting the feature set comprises converting the feature set, by a feature analytics engine, into the score that corresponds to the medical condition of the user.

14. The method as in claim 13, wherein the feature analytics engine comprises a fully connected regression network configured to convert the feature set into the score.

15. The method as in claim 13, wherein the feature analytics engine comprises a decision tree configured to map the feature set into the score.

16. A wearable device, the wearable device comprising:
at least one memory;
at least one processor coupled to the at least one memory; and
a set of electronic sensors coupled to the at least one processor, the set of electronic sensors configured to detect user movements of a user and to generate signal data representing the user movements, and wherein the at least one processor is configured to:
inputting the signal data into a model to identify a feature set, and
converting the feature set into a score that corresponds to a medical condition of the user.

17. The wearable device of claim 16, wherein the least one processor is further configured to prompt the user to perform at least one predetermined exercise before generating the signal data.

18. The wearable device of claim 17, wherein the at least one predetermined exercise the user is prompted to perform comprises at least one exercise to performed with an extremity of the user on which the wearable device is worn.

19. The wearable device of claim 18, the least one processor is further configured to generate the signal data while the user performs the at least one exercise with the extremity of the user on which the wearable device is worn.

20. The wearable device of claim 16, wherein:
the user movements include finger tapping of a thumb against an index finger on a same hand of the user on which the wearable device is worn.

21. The wearable device of claim 20, wherein the score corresponds to a rating on a unified Parkinson's disease rating scale.

22. The wearable device of claim 16, wherein the wearable device comprises a wristband.

23. The wearable device of claim 16, wherein the model comprises a convolutional neural network (CNN) configured to receive the signal data as input and to identify the feature set as output.

24. The wearable device of claim 16, wherein the set of electronic sensors comprises an inertial measurement unit (IMU) sensor.

25. The wearable device of claim 24, wherein the set of electronic sensors includes a photoplethysmography (PPG) sensor.

26. The wearable device of claim 16, wherein converting the feature set comprises converting the feature set, by a feature analytics engine, into the score that corresponds to the medical condition of the user.

27. A non-transitory storage medium comprising code that, when executed by processing circuitry, causes the processing circuitry to perform a method, the method comprising:
detecting, by a wearable device worn by a user, user movements of the user;
generating, by the wearable device, signal data representing the user movements;
inputting the signal data into a model to identify a feature set; and
converting the feature set into a score that corresponds to a medical condition of the user.

28. The non-transitory storage medium of claim 27, wherein:
the wearable device comprises a wristband;
the model comprises a convolutional neural network (CNN) configured to receive the signal data as input and to identify the feature set as output;
the wearable device includes a set of electronic sensors, each of the set of electronic sensors being configured to produce the signal data in response to detecting the user movements; and
the set of electronic sensors comprises an inertial measurement unit (IMU) sensor and a photoplethysmography (PPG) sensor.
